(19) United States
(12) Patent Application Publication
(10) Pub. No.: US 2011/0294692 A1
(43) Pub. Date: Dec. 1, 2011

(54) USE OF MIR-126 FOR ENHANCING HEMATOPOIETIC STEM CELL ENGRAFTMENT, FOR ISOLATING HEMATOPOIETIC STEM CELLS, AND FOR TREATING AND MONITORING THE TREATMENT OF ACUTE MYELOID LEUKEMIA

(76) Inventors: John Dick, Toronto (CA); Eric Lechman, Toronto (CA); Kristin Hope, Toronto (CA); Hidefumi Hiramatsu, Toronto (CA)

(21) Appl. No.: 13/128,119

(22) PCT Filed: Nov. 6, 2009
(86) PCT No.: PCT/CA2009/001612

§ 371 Date: Jul. 29, 2011

Related U.S. Application Data
(60) Provisional application No. 61/112,971, filed on Nov. 10, 2008.

(51) Int. Cl. C40B 30/04; C12Q 1/68

(52) U.S. Cl. 506/9; 435/6.12; 435/6.1

(57) ABSTRACT

There is disclosed herein composition, methods and uses relating to miR-126 as a measure of engraftment potential of a population of hematopoietic stem cells (HSCs), as a method of purifying HSCs and in the monitoring or treatment of acute myeloid leukemia.

### FIELD OF THE INVENTION

The invention relates to methods and uses in respect of miR-126 in the purification of acute myeloid leukemia (AML) stem cells and normal hematopoietic stem cells. The invention also relates to the diagnosis and treatment of AML by providing a novel biomarker for screening the bone marrow and peripheral blood of leukemia patients. The invention also relates to enhanced stem cell transplantation by providing a novel biomarker for the identification of human umbilical cord blood (CB), bone marrow and peripheral blood stem cells.

### BACKGROUND

Leukemia stem cells (LSCs) are a biologically distinct blast population positioned at the apex of the acute myeloid leukemia (AML) developmental hierarchy. A more complete understanding of the unique properties of LSCs is crucial for the identification of novel AML regulatory pathways and the subsequent development of innovative therapies that effectively target these cells in leukemia patients. Typically, studies overlook the heterogeneity of AML and the existence of LSC, potentially masking important molecular pathways.

MicroRNAs (miRNAs) are an emerging class of non-coding small RNAs that negatively regulate the expression of protein-encoding genes. Normal miRNA expression is tissue and developmental stage restricted, suggesting important roles in tissue specification and/or cell lineage determination (Abbott et al., 2005)(Brennecke et al., 2003)(Chen et al., 2004)(Chen, 2005)(Xu et al., 2003). Several studies have already demonstrated that miRNA expression levels are dysregulated in AML. However, little is known of the contribution of miRNAs to the regulation of gene expression and maintenance of LSCs. Progress has been limited in the pursuit for the enhanced purification of leukemia stem cells in part due to heterogeneity in the expression of cell surface markers.

Elderly patients with acute leukemia and poor risk cytogenetics have a median survival of less than one year. Thus, for these patients and those with relapsed refractory disease novel therapies are needed. As many of these patients are frail, therapies that achieve an anti-leukemia effect without significant toxicity are highly desirable. The transplantation of human hematopoietic stem cells (HSC) from bone marrow of CB has been one of the most important clinical applications of stem cell biology. HSC transplantation in individuals with leukemia enables the use of high dose chemotherapy regimen and subsequent HSC rescue to overcome the hematopoietic failure due to chemotherapy, enhancing cure rate for hematologic malignancy.

Although HSC transplantation is a well developed therapeutic, future enhancements of HSC transplantation such as gene therapy, purging, purification are impaired due to the absence of reliable cell surface markers that can be used for HSC identification and purification. The standard marker used clinically is CD34; however cell populations isolated with this cell surface marker contain large numbers of progenitors and other non-HSC. The purity of HSC is only 0.01% in this fraction as tested on the basis of the gold standard assay for human HSC that involves repopulation of NOD/SCID mice. It is very difficult to find markers that purify HSC to homogeneity. As well, cell surface markers are often differentiation markers making it hard to find HSC specific markers; often, HSC selection is based on combinations of what the HSC does not express (negative sorting), making it hard to use clinically. There is a great need for a HSC specific marker that isolates HSC on the basis of some biological function. With the discovery of the miRNA axis of regulation, this is an important area to explore of functional markers.

Although a large portion of the genome is actively transcribed, only 1.2% of the human genome encodes protein. The remaining 98% of the transcribed output of the human genome consists of non-protein coding RNAs (ncRNAs). The majority of these RNAs derive from the introns of protein coding genes and the exons and introns of non-protein coding genes (Mattick, 2001; Mattick, 2003). MicroRNAs (miRNAs) are a newly discovered class of ncRNAs that appear to be involved in diverse biological processes. With greater than 500 identified members per species in higher eukaryotes, miRNAs represent one of the largest gene families identified. Many miRNAs display conservation across related species, supporting the idea that miRNA control is a general mechanism of cell regulation. miRNA expression is tissue and developmental stage restricted, suggesting important roles in tissue specification and/or cell lineage determination. Thus far, miRNAs have been implicated in the regulation of diverse processes including control of developmental timing, cell cycle control, hematopoietic cell differentiation, apoptosis, fat metabolism and insulin secretion, and organ development (Lau et al., 2001b)(Xu et al., 2003)(Chen et al., 2004)(Bashirullah et al., 2003). Genomic annotation of miRNAs shows that most are located in defined transcription units, especially within intronic regions of known genes in the sense or anti-sense orientation (Lau et al., 2001a) (Lagos-Quintana et al., 2001b). About 92 out of 232 miRNAs were found in the introns of protein coding gene and 27 in non-protein coding genes (Rodriguez et al., 2004). An additional, thirty miRNAs were found to overlap with exons of non-protein coding genes while 24 miRNAs considered mixed depending on alternative splicing patterns (Rodriguez et al., 2004). About 50% of all known miRNAs were found in close proximity to other miRNAs and expressed as polycistronic primary transcripts (Lau et al., 2001a; Lagos-Quintana et al., 2001a; Cai et al., 2004). Transcription is mediated by RNA polymerase II, with the pri-miRNA transcript containing a cap structure and poly A tail typical of mRNAs (Cai et al., 2005).

miRNAs are processed in a two-step cleavage process from longer primary transcripts that have been termed pri-miRNAs. These transcripts are processed by the RNase III endonuclease Drosha in the nucleus of mammalian cells (Lee et al., 2003). Drosha is part of the microprocessor complex consisting of Drosha and a double-stranded RNA binding protein the Digeorge syndrome critical region gene 8 (DGCR8) (Han et al., 2004; Denli et al., 2004; Gregory et al., 2004; Landthaler et al., 2004). The microprocessor complex cleaves RNA hairpins that contain a large terminal loop of approximately two helical turns to excise 65-75 nt precursors called a pre-miRNA (Zeng and Cullen, 2005). The pre-miRNAs are then exported from the nuclease by Exportin 5 and processed by the cytoplasmic RNase III endonuclease Dicer 1 into 22 bp duplexes with a 2 nt overhang at the their 3′ ends (Lund et al., 2004) (Yi et al., 2003; Bernstein et al., 2001). Dicer 1 processed short duplex RNAs are incorporated in the miRISC complex which contains an Argonaut family member and the fragile X mental retardation protein (FMRP) (Lee et al., 2004) (Jin et al., 2004). Only one strand of the processed duplex is retained in the miRISC complex. Strand selection is determined by relative stability of the two ends of the duplex, favoring the one whose 5′ end is less tightly paired (Khvorova et al., 2003). The miRISC complex targets mRNAs by binding to sequences that are imperfectly complementary to the miRNA leading to translational repression by a yet unidentified mechanism. Biogenesis of miRNAs seems to be regulated on two levels. The main mechanism seems to be transcriptional control perhaps through the temporal regulatory element (TRE) which is situated upstream of several miRNAs (Johnson et al., 2003). Some miRNAs may be controlled at the post-transcriptional level. For example miR-39 precursor is expressed ubiquitously in C. elegans, but mature miR-39 is only expressed in the embryo (Ambros et al., 2003).

Several recent lines of evidence strongly suggest a role for miRNAs in stem cell maintenance and proliferation. Embryonic stem (ES) cell specific miRNAs were cloned from both murine and human lines. A total of 15 ES cell specific miRNAs were revealed by comparing murine undifferentiated and differentiated ES cells (Houbaviy et al., 2003). Interestingly, 6 of these candidates were found to be clustered together and specific for mouse trophoblastic stem cells.

The functional importance of miRNA expression in stem cells was elucidated when targeted knockout of Dicer within ES cell lines induced both cell division and proliferation defects (Murchison et al., 2005). Furthermore, transgenic mice derived from Dicer-deficient ES cells die at embryonic day 7.5 with embryos devoid of Oct 4/brachyury positive multipotent stem cells (Bernstein et al., 2003). Drosophila germ line stem cell Dicer-1 mutants revealed that the miRNA pathway is essential for stem cell division and for cell cycle G1/S checkpoint bypass. In addition, conditional tissue specific Dicer knockouts confirmed the essential role of miRNAs for morphogenesis of the skin (Andl et al., 2006) lung epithelium (Harris et al., 2006) and the vertebrate limb (Harfe et al., 2005). Since Dicer is responsible for siRNA and miRNA biogenesis, it was thought that some of the observed stem cell effects may be due to loss of centromeric silencing rather than compromised miRNA production. However, ES cell knockouts of DGCR8, a double-stranded RNA binding protein with no other known functions, demonstrated that miRNAs are essential for silencing ES cell self-renewal. These studies also demonstrated the absolute requirement of DGCR8 for the biogenesis of miRNAs (Wang et al., 2007).

Some miRNAs have already been shown to play important roles in the differentiation and lineage determination of hematopoietic cells. For example, miR-181a was found to be expressed preferentially in B-cells. Ectopic expression of miR-181a in hematopoietic precursor cells resulted in a 2-fold increase in B lineage cells (Chen et al., 2004). In addition, miR-142s and miR-223 were found to be expressed in B and myeloid cells respectively, however, enforced expression of both lead to an increase of 30-50% in T cells compartment (Chen et al., 2004). Furthermore, miR-223 was found to maintain granulocytic differentiation by targeting a negative regulator of C/EBPα (Fazi et al., 2005). Two additional miRNAs, miR-221 and miR-222 appear to be down-regulated in erythroid differentiation. Target prediction algorithms suggested c-kit was targeted by both miRNAs and expression studies indicated an inverse correlation of c-kit and miR-221/miR-222 expression. Luciferase based assays confirmed that c-kit was a target of miR-221 and mir-222, suggesting that unblocking of the translational repression of c-kit was an important event in erythropoiesis (Felli et al., 2005). Finally, conditional dicer knockout in T cells revealed a reduced viability of mature T cell populations and aberrant cytokine production of T-helper cells (Cobb et al., 2005)(Muljo et al., 2005).

miRNAs may also play a critical role in inducing and maintaining the leukemogenic state. Many characterized miRNAs are located at fragile sites, minimal loss of heterozygosity regions, minimal regions of amplification or common breakpoint regions in human cancers (Calin et al., 2004). For example, chromosomal translocation t(8; 17) in an aggressive B-cell leukemia results in fusion of miR-142 precursor and a truncated MYC gene (Gauwerky et al., 1989). Furthermore, both miR-15 and miR-16 are located within a 30 kb deletion in CLL, and in most cases of this cancer both genes are deleted or under-expressed (Calin et al., 2002). Recently, miR-15 and miR-16 levels were found to be inversely correlated to BCL2 levels in CLL and that both miRNAs negatively regulate BCL2 at the post-transcriptional level (Cimmino et al., 2005). Enforced expression of miR-15 and miR-16 in leukemia cell lines induced apoptosis (Cimmino et al., 2005). In addition, mice transplanted with hematopoietic stem cells (HSC) over-expressing both c-Myc and the miR-17-92 polycistron developed cancers earlier with a more aggressive nature when compared to lymphomas generated by c-myc alone (He et al., 2005). Also, mir-155 is located in the final exon of the B-cell integration cluster (BIC), a noncoding RNA originally identified as a transcript derived from a common retroviral insertion site in avian leukosis virus-induced lymphoma cells in birds (Tam et al., 1997). The final exon was later shown to accelerate Myc-mediated lymphomagenesis in a chicken model definitively demonstrating the tumour-promoting activity of mir-155 (Tam et al., 2002). In line with this finding, mir-155 over-expression has regularly been observed in human B cell lymphomas (E is et al., 2005). Finally, over-expression of miR-155 in the B-cell compartment of transgenic mice induced a B-ALL like disease (Costinean et al., 2006).

There remains a need to further identify the relationship between miRNAs and HSC function and malignant hematopoiesis.

### SUMMARY OF THE INVENTION

In accordance with one aspect, there is provided a method for identifying the engraftment potential of a population of hematopoietic stem cells (HSCs) comprising determining the relative level of miR-126 in the population, wherein the relative level of miR-126 in the population is indicative of engraftment potential.

In accordance with a further aspect, there is provided a method for identifying the engraftment potential of a fraction from a population of HSCs comprising sorting the population of HSCs into fractions and determining the relative level of miR-126 in one or more fractions, wherein the relative level of miR-126 in the population is indicative of engraftment potential.

In accordance with a further aspect, there is provided a method for the increasing engraftment potential of a population of HSCs to be administered to a patient comprising, sorting the population of HSCs into fractions and selecting fractions exhibiting increased levels of miR-126 expression for administration to the patient.

In accordance with a further aspect, there is provided a method for purifying HSCs from a population of cells comprising sorting the population of cells into fractions and selecting fractions exhibiting increased levels of miR-126 expression.

In accordance with a further aspect, there is provided a method for monitoring the treatment or progression of acute myeloid leukemia in a patient comprising isolating a population of AML blast cells, determining the level of miR-126 in the AML blast cells and comparing the level of miR-126 to a previous level of miR-126 in AML blast cells, wherein a reduction in the level of miR-126 is indicative that the patient's acute myeloid leukemia is ameliorating.

In accordance with a further aspect, there is provided an expression vector comprising the coding sequence for miR-126 operably linked to an expression control sequence and a cultured cell with said vector.

In accordance with a further aspect, there is provided a method for treating a patient having acute myeloid leukemia comprising modulating the level of miR-126 in leukemia stem cells and progenitor cells in the patient.

In accordance with a further aspect, there is provided use of a therapeutically effective amount of miR-126 in the treatment of acute myeloid leukemia, or in the preparation of a medicament therefor.

In accordance with a further aspect, there is provided use of a therapeutically effective amount of a vector described herein in the treatment of acute myeloid leukemia, or in the preparation of a medicament therefor.

In accordance with a further aspect, there is provided a composition comprising miR-126 and a pharmaceutically acceptable carrier for treating a patient having acute myeloid leukemia.

In accordance with a further aspect, there is provided a composition comprising a vector described herein and a pharmaceutically acceptable carrier for treating a patient having acute myeloid leukemia.

### BRIEF DESCRIPTION OF THE FIGURES

FIG. 1 shows a schematic for the high speed sorting of distinct developmental sub-compartments of primary human AML patient samples.

FIG. 2 shows a schematic outlining the functional evaluation and gene expression analysis of enriched developmental sub-compartments of AML.

FIG. 3 shows functional evaluation of highly enriched AML stem/progenitor cells sorted by CD34 and CD38 in both the in vitro (FIG. 3A) and in vivo (FIG. 3B) studies.

FIG. 4 shows the unsupervised cluster analysis of miRNA expression.

FIG. 5 shows the t-test results yielding a specific LSC/progenitor miRNA signature.

FIG. 6 illustrates the quantitative real time PCR validation of miR-126 (A) qRT-PCR results showing that miR-126 is most highly expressed in the CD34+CD38−(LSC-enriched) fraction of a primary AML patient sample with miR-126* most highly expressed in the CD34−CD38+ compartment. (B) qRT-PCR results showing that miR-126 is most highly expressed in the CD34+CD38− compartment of 2 sort primary AML patient samples and within lin-CB.

FIG. 7 is a schematic outlining the genomic organization of the miR-126 gene.

FIG. 8A shows high-speed sorting of the AML 8227 cell line characterized by a long-term in vitro maintenance of an AML phenotypic and functional hierarchy based on CD34/CD38 cell surface staining. FIG. 8B is a graph showing the culture initiating potential of the four sub-populations sorted from the parent culture in FIG. 8A.

FIGS. 9A-C generally relate to the in vitro biosensor-mediated detection of miR-126-3p expression in the AML 8227 cell line; (A) a schematic of the Bd.LV.mirT biosensor lentivirus construct; (B) flow cytometry evaluation of gated cells transduced with control or miR-126 biosensor lenti-vectors and (C) a graphical representation of the calculated fold eGFP repression.

FIGS. 10A and B illustrate the in vivo biosensor-mediated expression of miR-126 in primary AML after engraftment in a NOD/SCID mouse; (A) flow cytometry evaluation of miR-126 sensor vector and control (top) expression in AML patient sample and (B) a graphical representation of the levels of miR-126 mediated eGFP repression.

FIGS. 11A-C illustrate the in vivo biosensor-mediated detection of miR-126 expression in primary human CB after engraftment in a NOD/SCID mouse.

FIG. 12A is a schematic showing the structure of antagomirs.

FIG. 12B are FACS plots showing antagomir-mediated knockdown of miR-126 within Bd.LV.miR-126-3pT transduced lin-CB.

FIG. 13A illustrates the FACS sorting scheme for the prospective isolation of human HSC from long-term in vitro culture of lin-CB using the Bd.LV miR-126-3pT reporter vector.

FIG. 13B are graphs showing the colony numbers and types generated after methylcellulose plating of eGFP^{high} and eGFP^{low} subpopulations of cultured lin—CB. E: erythroid, G: granulocytic, M: macrophage, GM: granulocytic/macrophage, GEMM: granulocytic/erythroid/megakaryocyte/macrophage.

FIG. 13C is a descriptive table summarizing the results of four independent prospective isolation experiments demonstrating that human HSC are contained within the miR-126^{high} fraction of the culture.

FIG. 14A illustrates the FACS sorting scheme for the prospective isolation of AML stem cells from Bd.LV miR-126-3pT transduced bulk AML xenografted into immunodeficient mice.

FIG. 14B shows a schematic of the analysis of immunodeficient mice xenotransplanted with the sorted subpopulations of each Bd.LV miR-126-3pT labeled AML

FIG. 14C is a descriptive table summarizing the results of four independent AML stem cell prospective isolation experiments showing that the LSC's are contained within a single miR-126^{high} or miR-126^{Int} gated population for each AML.

### DETAILED DESCRIPTION

In the following description, numerous specific details are set forth to provide a thorough understanding of the invention. However, it is understood that the invention may be practiced without these specific details

As used herein, a “cultured cell” means a cell which has been maintained and/or propagated in vitro. Cultured cells include primary cultured cells and cell lines. As used herein, “culturing the cell” means providing culture conditions that are conducive to polypeptide expression. Such culturing conditions are well known in the art.

As used herein “engrafting” a stem cell, preferably an expanded hematopoietic stem cell, means placing the stem cell into an animal, e.g., by injection, wherein the stem cell persists in vivo. This can be readily measured by the ability of the hematopoietic stem cell, for example, to contribute to the ongoing blood cell formation.

As used herein “hematopoietic stem cell” refers to a cell of bone marrow, liver, spleen or cord blood in origin, capable of developing into any mature myeloid and/or lymphoid cell.

It is also contemplated that the peptides of the invention may exhibit the ability to modulate biological, such as intracellular, events. As used herein “modulate” refers to a stimulatory or inhibitory effect on the biological process of interest relative to the level or activity of such a process in the absence of a peptide of the invention.

As used herein, the “nucleic acid molecule” means DNA molecules (e.g., a cDNA) and RNA molecules (e.g., an mRNA) and analogs of the DNA or RNA generated, e.g., by the use of nucleotide analogs. The nucleic acid molecule can be an oligonucleotide or polynucleotide and can be single-stranded or double-stranded.

As used herein “operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence. Similarly, “control elements compatible with expression in a subject” are those which are capable of effecting the expression of the coding sequence in that subject.

As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

In accordance with one aspect, there is provided a method for identifying the engraftment potential of a population of hematopoietic stem cells (HSCs) comprising determining the relative level of miR-126 in the population, wherein the relative level of miR-126 in the population is indicative of engraftment potential.

In accordance with a further aspect, there is provided a method for identifying the engraftment potential of a fraction from a population of HSCs comprising sorting the population of HSCs into fractions and determining the relative level of miR-126 in one or more fractions, wherein the relative level of miR-126 in the population is indicative of engraftment potential.

In accordance with a further aspect, there is provided a method for the increasing engraftment potential of a population of HSCs to be administered to a patient comprising, sorting the population of HSCs into fractions and selecting fractions exhibiting increased levels of miR-126 expression for administration to the patient.

In accordance with a further aspect, there is provided a method for purifying HSCs from a population of cells comprising sorting the population of cells into fractions and selecting fractions exhibiting increased levels of miR-126 expression.

In certain embodiments of the methods described herein, the population of cells or HSCs is sorted using biological markers, preferably, selected from the group consisting of CD34, CD38, CD90 and CD45RA.

Preferably, the fraction exhibiting increased levels of miR-126 expression is a CD34+ fraction. Further, the fraction is additionally CD38−, CD90+ and CD45RA−, in increasing preferability, independently or in combination.

In accordance with a further aspect, there is provided a method for monitoring the treatment or progression of acute myeloid leukemia in a patient comprising isolating a population of AML blast cells, determining the level of miR-126 in the AML blast cells and comparing the level of miR-126 to a previous level of miR-126 in AML blast cells, wherein a reduction in the level of miR-126 is indicative that the patient's acute myeloid leukemia is ameliorating.

In accordance with a further aspect, there is provided an expression vector comprising the coding sequence for miR-126 operably linked to an expression control sequence.

In accordance with a further aspect, there is provided a cultured cell comprising the vectors described herein.

In accordance with a further aspect, there is provided a method for treating a patient having acute myeloid leukemia comprising modulating the level of miR-126 in leukemia stem cells and progenitor cells in the patient. Preferably, the modulating is increasing. Also preferably, the modulating the level of miR-126 comprises administering a therapeutically effective amount of miR-126 to the patient.

In certain embodiments, the modulating the level of miR-126 comprises administering a therapeutically effective amount of a vector described herein.

In accordance with a further aspect, there is provided use of a therapeutically effective amount of miR-126 in the treatment of acute myeloid leukemia, or in the preparation of a medicament therefor.

In accordance with a further aspect, there is provided use of a therapeutically effective amount of a vector described herein in the treatment of acute myeloid leukemia, or in the preparation of a medicament therefor.

In accordance with a further aspect, there is provided a composition comprising miR-126 and a pharmaceutically acceptable carrier for treating a patient having acute myeloid leukemia.

In accordance with a further aspect, there is provided a composition comprising a vector described herein and a pharmaceutically acceptable carrier for treating a patient having acute myeloid leukemia.

The present invention is further illustrated by the following examples. The examples and their particular details set forth herein are presented for illustration only and should not be construed as a limitation on the claims of the present invention.

### EXAMPLES

### Example 1

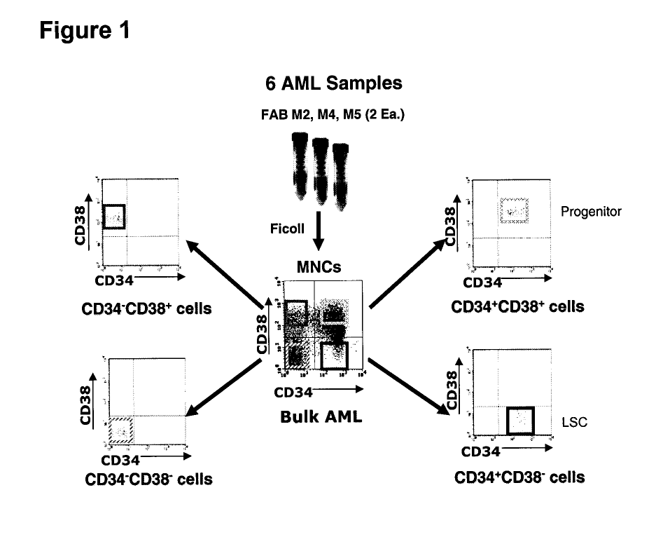

FIG. 1 illustrates a schematic for the high speed sorting of distinct developmental sub-compartments of primary human AML patient samples.

Peripheral blood cells were collected from patients with newly diagnosed AML after obtaining informed consent according to procedures approved by the Research Ethics Board of the University Health Network. Individuals were diagnosed according to the standards of the French-American-British classification. Cells from six different samples representing 3 AML subtypes were investigated in our studies. Specifically, low density peripheral blood cells were collected from 6 AML patients representing 3 FAB subtypes (2 M2, 2 M4 and 2 M5) by density centrifugation over a Ficoll gradient. Low-density mononuclear cells isolated from individuals with AML were frozen viably in FCS plus 10% (vol/vol) DMSO. For sorting of AML sub-populations, AML blasts were stained with anti-CD34−APC (Becton-Dickinson) and anti-CD38-PE (Becton-Dickinson) and were sorted using a Dako Mo-Flo™ (Becton-Dickinson) cell sorter. Viability and purity of each subpopulation exceeded 95%. Fractionated cells were captured in 100% FCS and recovered by centrifugation. As a result, each AML patient sample was sorted into 4 subpopulations based upon CD34 and CD38 antibody staining and cells recovered for functional and gene expression analysis.

### Example 2

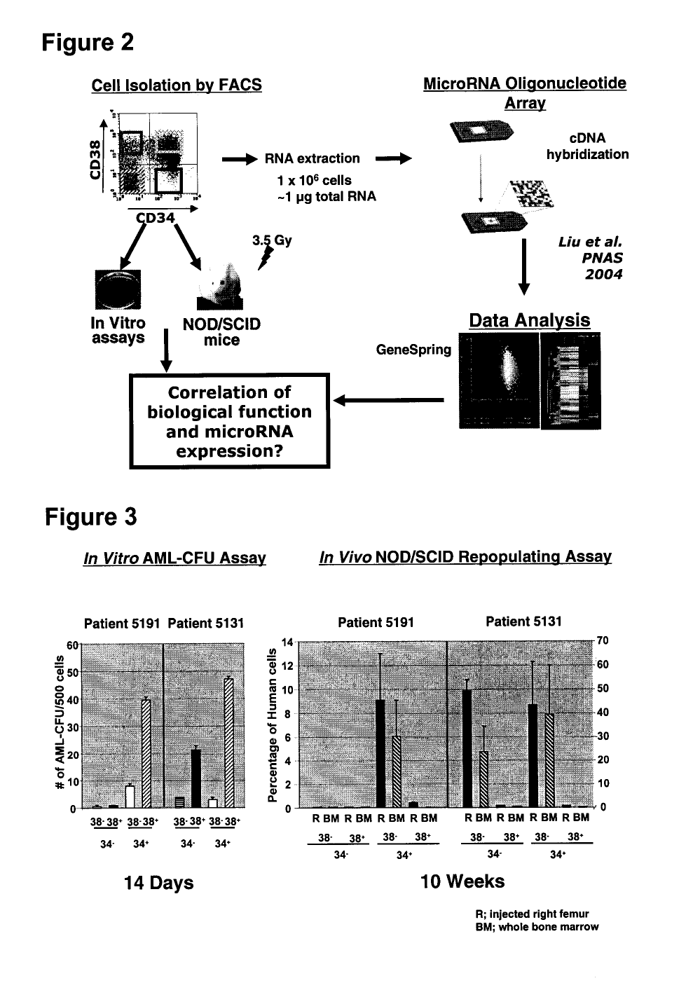

FIG. 2 illustrates a schematic for the functional evaluation and gene expression analysis of enriched developmental sub-compartments of AML. Using this approach, a correlation between biological function and miRNA expression could be established. In summary, the functional characteristics of recovered post-sort AML sub-populations were assayed in serum-free liquid culture for proliferative potential, in colony forming assays for progenitor activity and by intra-femoral transplantation into sub-lethally irradiated NOD/SCID immuno-deficient mice for SL-IC(SCID-Leukemia initiating cell or LSC) activity. In addition, RNA was extracted from each sub-population and first strand synthesis performed using a biotin labeled poly A primer. After synthesis, RNA/DNA hybrids were denatured and the RNA template degraded. Biotin labeled targets were hybridized onto miRNA array chips, washed and detected. Chips were scanned and analyzed using the GENESPRING software.

### Detailed Material and Methods

Primary AML patient samples were collected and sorted into 4 sub-populations (see Example 1).

Suspension Culture assays. Suspension cultures were initiated with sorted AML cells at 10⁵ cells/mL in serum-free media (SFM) consisting of X-VIVO 10 (BioWhittaker) containing 10 μg/mL insulin, 200 μg/mL transferrin, 2% Bovine serum albumin and a cocktail of recombinant growth factors including 3 U/mL recombinant human erythropoietin, 20 ng/mL rh IL-3, 20 ng/mL rh IL-6, 20 ng/mL rh G-CSF, 20 ng/mL rh GM-CSF, 100 ng/mL rh SCF and 100 ng/mL FLT3L. Cultures were established in BD Falcon™ non-tissue culture treated 24 well suspension plates and maintained at 37° C. in a 5% CO₂ humidified incubator. Cells were passaged by replating 10⁵ viable cells weekly. Suspension cells were maintained for 6 weeks or until cells displayed negative proliferation for two weeks. Viable cells were counted by hemocytometer in the presence of trypan blue and assessed for progenitor activity in colony forming assays described below (Ailles et al., 1997).

### Blast Colony Assays (CFU-Blast)

FACS-sorted AML sub-populations were plated immediately after sorting and once weekly from the AML suspension cultures in α-methylcellulose culture medium containing 15% fetal calf serum (FCS), 15% human plasma, 48 μM β-mercaptoethanol, 20 μM glutamine, 1% bovine serum albumin and the growth factor cocktail described above for suspension cultures. After 14 days of incubation in a humidified 37° C. incubator with 5% CO₂, blast clusters (10-20 cells) and colonies (>20 cells) were counted under an inverted microscope and the numbers pooled to obtain CFU-blast counts (Ailles et al., 1997).

Transplantation of Sorted AML Cells into NOD/SCID Mice

NOD/SCID mice (Jackson Laboratory, Bar Harbor, Me.) were bred and maintained in microisolater cages. Twenty-four hours before transplantation, mice were irradiated with 3 Gy γ irradiation from a ¹³⁷Cs source. Sorted AML cells were counted and resuspended into 1% FCS in 1× phosphate buffered saline (PBS) pH 7.4 and injected directly into the right femur of each experimental animal. Eight to ten weeks post-transplant, mice were euthanized by cervical dislocation and hind leg bones removed and flushed with media to recover engrafted cells. Percent human AML engraftment was assessed by flow cytometry for human CD45+ staining cells (Lapidot et al., 1994).

miRNA Array

On average, 10⁶ recovered cells were placed into 1 mL Trizol® reagent for RNA isolation. RNA was recovered by adding 0.2 mL chloroform followed by a short incubation for 3 minutes at RT. Samples were centrifuged at 12,000×g for 15 minutes. The aqueous phase was transferred to a fresh tube and 0.5 mL of isopropyl alcohol added followed by a ten minute incubation at RT. Samples were centrifuged for 10 minutes at 12,000×g. and the supernatant was removed, pellet washed with 75% ethanol, and recovered by a 7,500×g spin for 5 minutes. RNA was briefly air dried and resuspended in nuclease free water. RNA concentration was determined by optical density measurement on a spectrophotometer. Five micrograms of total RNA were separately added to reaction mix in a final volume of 12 μL containing 1 μg of [3′-(N)8-(A)12-biotin-(A)12-biotin-5′] oligonucleotide primer. The mixture was incubated for 10 min at 70° C. and chilled on ice. With the mixture remaining on ice, 4 μL of 5× first-strand buffer, 2 μL of 0.1 M DTT, 1 mL of 10 mM dNTP mix, and 1 μL of superscript II RNaseH—reverse transcriptase (200 units/μL) was added to a final volume of 20 μL, and the mixture was incubated for 90 min in a 37° C. water bath. After the incubation for first-strand cDNA synthesis, 3.5 μL of 0.5 M NaOH/50 mM EDTA was added into 20 μL of first strand reaction mix and incubated at 65° C. for 15 min to denature the RNA/DNA hybrids and degrade RNA templates. Then, 5 μL of 1 M Tris-HCL (pH 7.6) was added to neutralize the reaction mix, and labeled targets were stored in 28.5 μL until chip hybridization.

Labeled targets for 5 mg of total RNA was used for hybridization on each Kimmel Cancer Center/Thomas Jefferson University miRNA microarray containing 368 probes in triplicate, corresponding to 245 human and mouse miRNA genes. All probes on the microarray are 40-mer oligonucleotides, spotted by contacting technologies and covalently attached to a polymeric matrix. The microarrays were hybridized in 6×SSPE (0.9 M sodium chloride/60 mM sodium phosphate/8 mM EDTA, pH 7.4)/30% formamide at 25° C. for 18 hours, washed in 0.75×TNT (Tris-HCL/sodium chloride/Tween 20) at 37° C. for 40 min, and processed by using direct detection for the biotin-containing transcripts by streptavidin-Alexa647 conjugate. Processed slides were scanned by using a PerkinElmer ScanArray XL5K Scanner with the laser set to 635 nm, at power 80 and PMT 70 settings, and a scan resolution of 10 mM (Liu et al., 2008).

### Data Analysis

Images were quantified by QUANTARRAY software by PerkinElmer. Signal intensities were calculated by subtracting local background from total intensities. Raw data was normalized and analyzed by GENESPRING software (version 6.1.1, Silicon Genetics, Redwood city, CA.) GENESPRING generates an average value of the three spot replicates of each miRNA, after data transformation, normalization was performed by using a per-chip, 50^{th} percentile method that normalizes each chip on its median, allowing comparison among chips. Hierarchical clustering for both genes and conditions were then generated by using standard correlation as a measure of similarity.

FIGS. 3A and 3B illustrates the biological features of highly enriched AML stem/progenitor cells. Patient samples were sorted based on CD34/CD38 expression pattern. Referring to FIG. 3A, for the in vitro study, each sub-fraction was placed into liquid culture and CFU formation was assessed weekly. Referring to FIG. 3B, for the in vivo study, purified AML populations were transplanted into non-lethally irradiated NOD/SCID mice and the percent of human 45+ cells in the femur (R) and bone marrow (BM) was determined at 10 weeks by flow cytometry. The CD34+/CD38− fraction of all 6 AML patient samples had NOD/SCID repopulating capacity. In addition, the CD34−/CD38− fraction of patient 5131 also retained SL-IC activity.

The in vitro data reveals that the majority of progenitor activity resides in the CD34+/CD38+ progenitor compartment for each AML sample. In addition, the data reveals the importance of functionally assessing each sorted subpopulation within in vitro and in vivo assays. Furthermore, referring to FIG. 3B, the in vivo data reveals that leukemic stem cell engraftment activity resides in the CD34+/CD38− compartment for each AML sample.

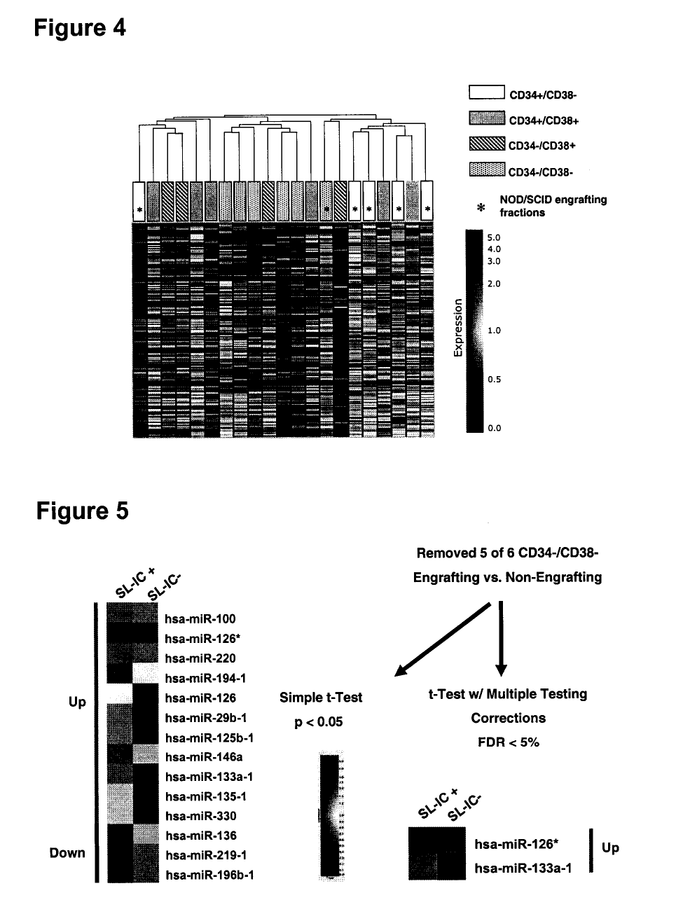

FIG. 4 illustrates the unsupervised cluster analysis of miRNA expression. Six AML patient samples were sorted into sub-fractions based upon CD34 and CD38 antibody staining. RNA was extracted from each sub-population and first strand synthesis as performed using a biotin labeled poly A primer. After synthesis, RNA/DNA hybrids were denatured and the RNA template degraded. Biotin labeled targets were hybridized onto miRNA array chips, washed and detected. Chips were scanned and analyzed using the GENESPRING software. Biotin labeled cDNA targets were hybridized onto miRNA array chips. Chips were scanned and analyzed using the GENESPRING software. Unsupervised cluster analysis revealed that fractions with similar biological function exhibit common miRNA expression profiles. For example, CD34+/CD38− NOD/SCID engrafting fractions of AML patient samples group closely together. This data suggests that specific miRNAs are preferentially expressed in AML stem cell enriched fractions.

FIG. 5 illustrates that supervised analysis yields a specific LSC/progenitor miRNA signature. In order to identify miRNAs differentially expressed in LSC/progenitor fractions of AML, bulk samples were removed and 5 of 6 CD34−/CD38− (normal erythroid, lymphoid populations) samples from the analysis. Sorted AML subpopulations with SL-IC(SCID/Leukemia-initiating cell) activity were then compared to non-engrafting fractions. A simple t-test yielded 14 candidate miRNAs with p values <0.05, 11 over-expressed and 3 under-represented in the SL-IC containing fractions. In order to further refine the candidate set, a more stringent t-test with an FDR (False Discovery Rate) of 5% was utilized. This test yielded a set of two miRNAs-hsa-miR-126* and hsa-miR-133a-1, both up-regulated in the SL-IC+ fractions. This approach therefore yielded a unique leukemia associated stem/progenitor signature.

### Example 3

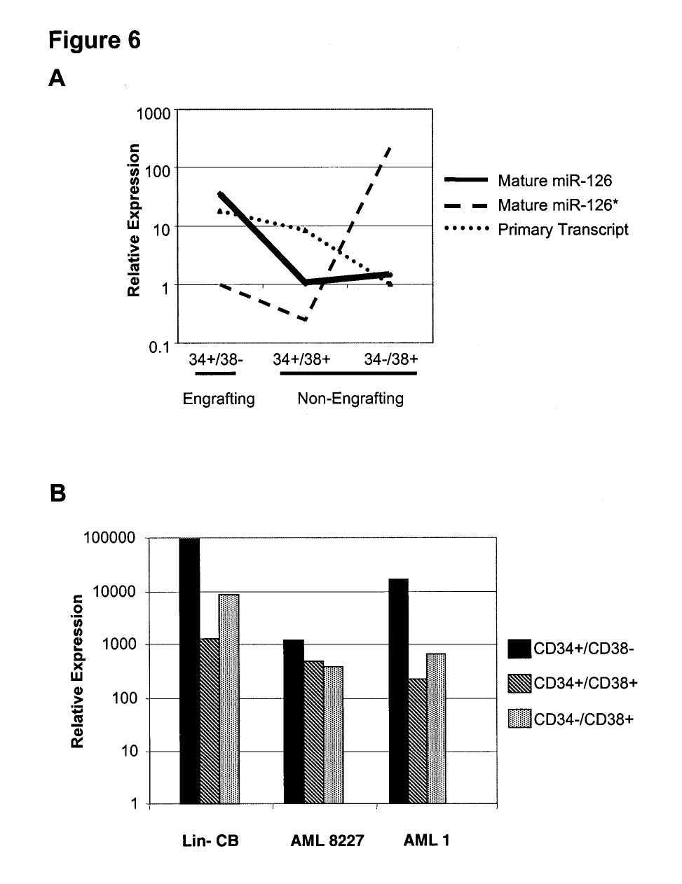

FIGS. 6A and 6B generally illustrate the quantitative real time PCR validation of miR-126.

For PCR validation of candidate miRNAs, ˜10⁵ sorted cells from primary AML and lin-CB were used to enrich for small RNA (>200 nt) using the mirVana™ kit (Ambion). Quantitative RT-PCR (qRT-PCR) expression analysis was performed by using SYBe®reen (Applied Biosystems) master PCR mix and mirVana™ qRT-PCR miRNA detection kits (Ambion) following the manufacturers instructions. Primer sets specific for hsa-miR-126, 126*, with U6 and 5S rRNA as positive controls. For each sample 25 ng of RNA was used. PCR was performed of an ABI7900 thermocycler (Applied Biosystems) and endpoint reactions products were also analyzed on a 3.5% high resolution agarose gel stained with ethidium bromide to discriminate between the correct amplification and the potential primer dimers.

Referring to FIG. 6A, qRT-PCR results are shown, revealing that miR-126 is most highly expressed in the CD34+CD38− (LSC-enriched) fraction of a primary AML patient sample with miR-126* most highly expressed in the CD34−CD38+ compartment.

FIG. 6B are qRT-PCR results showing that miR-126 is most highly expressed in the CD34+CD38− compartment of 2 sort primary AML patient samples and within lin-CB.

The data suggests the presence of a strand-specific miRNA hairpin processing bias within different developmental sub-compartments of AML. Overall levels of miR-126 are 10-100 fold lower in CD34+CD38− cells of leukemia compared to CD34+C38− of lin-CB. These results are consistent with previous data showing that the overall levels of most miRNAs are reduced in many cancers compared to their normal tissue counterpart. Indeed, enforced expression of miR-126 has been shown to reduce metastasis in several solid tumor animal models. Thus, it stands to reason that increasing endogenous levels of miR-126 in acute myelogenous leukemia stem and primitive progenitor cells may have therapeutic value.

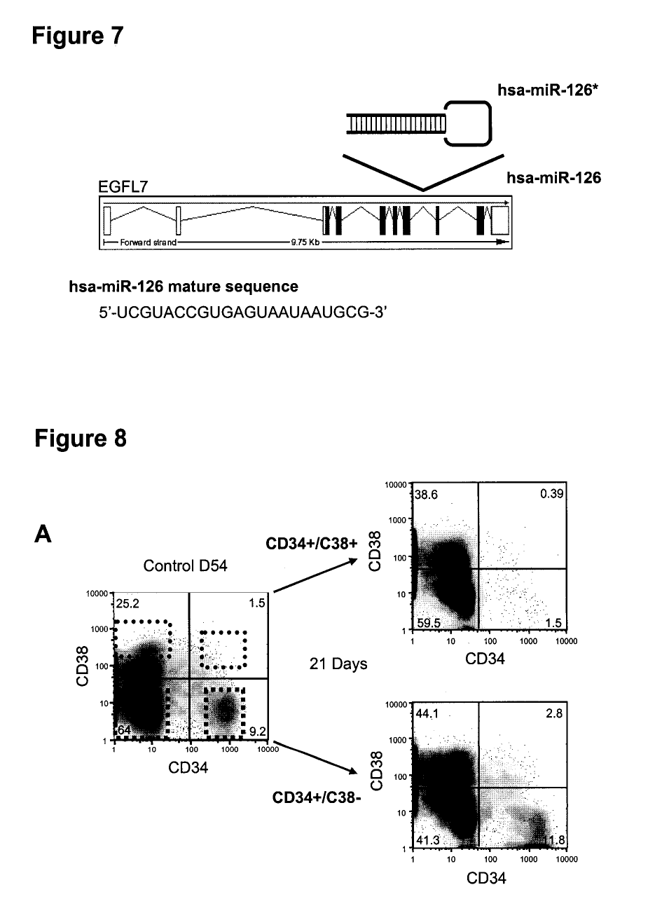

FIG. 7 illustrates a schematic outlining the genomic organization of the miR-126 gene. The hairpin encoding miR-126 is embedded within intron 7 of the EGFL7 protein coding gene. The hairpin encodes both miR-126 and miR-126*. The mature biologically active form of miR-126 is 22 nucleotides long and exerts its effects by binding to the 3′ untranslated regions of mRNA for protein coding genes. Mature miR-126 has the following sequence 5′-UCGUACCGUGAGUAAUAAUGCG-3′ (SEQ ID NO.1).

### Example 4

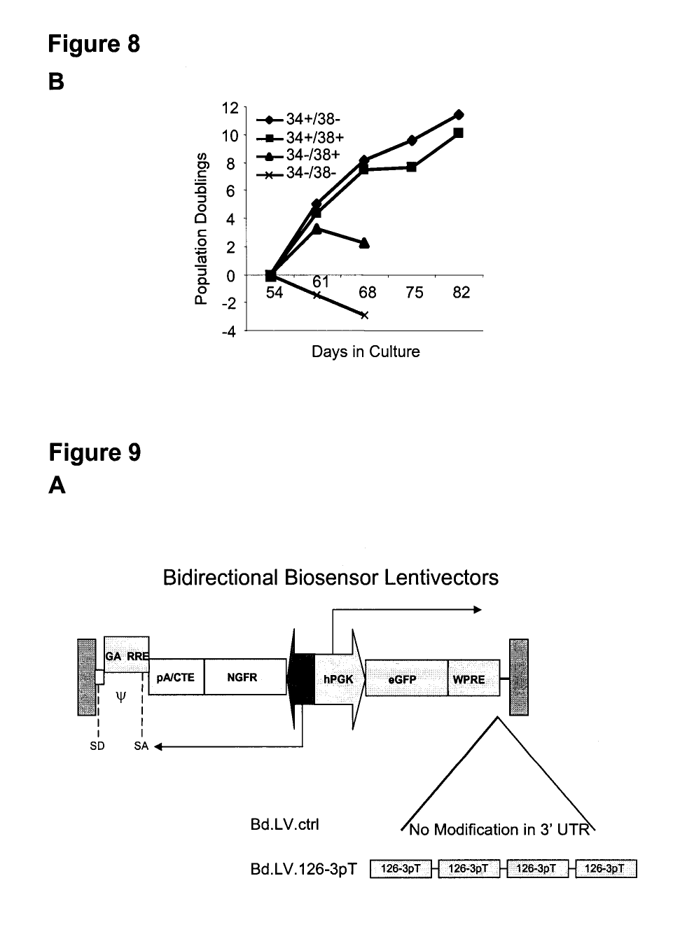

The studies with respect to FIGS. 8A and 8B utilized a cell line with long-term in vitro maintenance of AML phenotypic and functional hierarchy. A unique primary AML patient sample (8227) was identified that could be maintained in growth factor supplemented serum-free culture conditions for over 200 days.

FIG. 8A shows the high-speed sorting of subpopulations based on CD34/CD38 cell surface staining from day 54 of the parent culture. Recovered cells were seeded into serum-free culture conditions to evaluate the potential to initiate a new culture and also to recapitulate a phenotypic AML hierarchy. FIG. 8B is a graph showing the population doublings of the four sub-populations sorted from the parent culture.

The results show that CD34+/CD38− and CD34+/CD38+ subpopulations were both able to initiate and maintain new 8227 cultures creating a new in vitro model system to study the AML hierarchy.

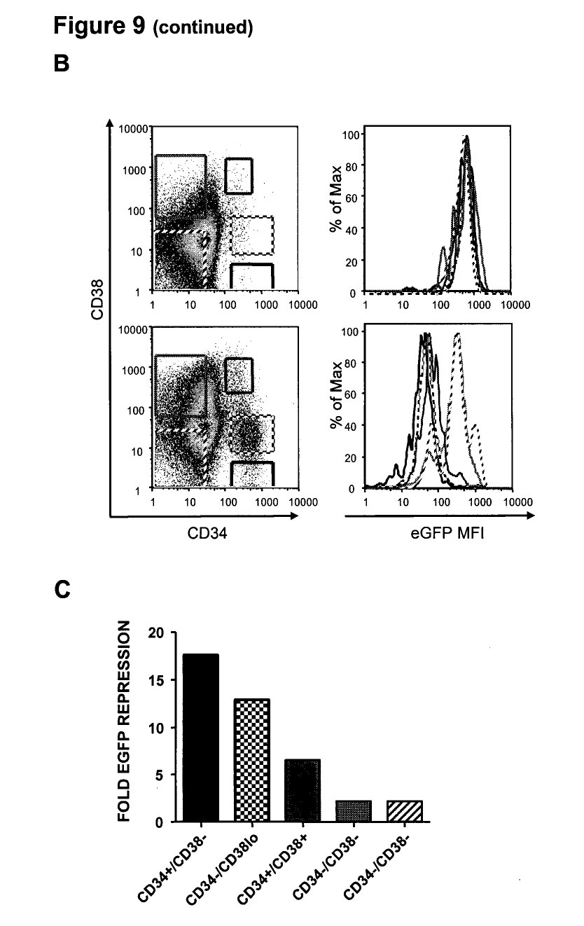

Generally, FIG. 9 illustrates the in vitro biosensor-mediated detection of miR-126-3p expression in AML 8227. FIG. 9A is a schematic of the Bd.LV.mirT biosensor lentivirus construct. Both empty control and miR-126-3pT biosensor lentivectors were kind gifts of Luigi Naldini. The bi-directional vectors are third generation lentiviral backbones with 4 tandem copies of a 23 bp sequence (mirT) with perfect complementarity to hsa-miR-126 into the 5′ untranslated region of an eGFP expression cassette driven by the ubiquitously expressed polyglycerol kinase promoter (hPGK). Viral supernatant was generated by transient transfection of 293T cells with packaging plasmids and pseudotyped with the vesicular stomatitis virus G protein as previously described (Guenecha et al. 2000). High titer stocks were prepared by ultracentrifugation and the function titers were determined by infection of HeLa cells and flow cytometry for ΔNGFR expression.

AML patient sample 8227 was transduced with a multiplicity of infection (MOI) of 50 with either the control or miR-126 biosensor vector in standard AML culture conditions (see Example 2). One week post transduction cells were harvested for flow cytometric analysis. Cells were stained with anti-CD34-PE, anti-CD38-PC5, anti-NGFR-APC antibodies and 4 color flow cytometry was performed on a FACSCalibur™ flow cytometer (Beckton Dickinson) with data analyzed by FlowJo 7.1 (Treestar, Inc). The mean fluorescence intensity was determined for both eGFP and NGFR for each gated subpopulation. The level of eGFP repression was determined by first calculating the transgene ratio (TGR): MFI (NGFR)/MFI (eGFP) for each gated population in control transduced and miR-126 biosensor transduced cells to normalize for viral integration. The fold eGFP repression is calculated by dividing TGR (Bd.LV.mirT)/TGR (Bd.LV.control).

FIG. 9B shows the flow cytometry evaluation and FIG. 9C is a graphical representation of the calculated fold eGFP repression.

The data suggests that biologically active miR-126 is expressed at much higher levels (17 fold eGFP repression vs. control vector) in the CD34+CD38− (LSC-enriched) population in vitro, while miR-126 activity declines along a gradient of further “differentiation” of the cells.

### Example 5

The in vivo biosensor-mediated expression of miR-126 in primary AML after engraftment in a NOD/SCID mouse was investigated.

Sorted AML 5131 CD34+CD38− cells were transduced with control and miR-126 Bd.LV.mirT lentivirus at an MOI of 50 for 48 hours in standard AML culture conditions (FIG. 2). A pre-transduction equivalent of 1×10⁵ cells were injected into preconditioned NOD/SCID mice as previously described in Example 2. Ten weeks later, mice were euthanized and bone marrow harvested for analysis. Human AML cells were enriched away from the murine bone marrow cells by negative selection. Murine depletion and AML cell enrichment were achieved by StemSep™ mouse/human chimera negative selection cocktail, according to the manufacturer's protocol (Stem Cell Technologies, Vancouver). Purified human AML cells were then stained with antibodies against CD34, CD38, and NGFR as previously described in Example 2 and analyzed by flow cytometry.

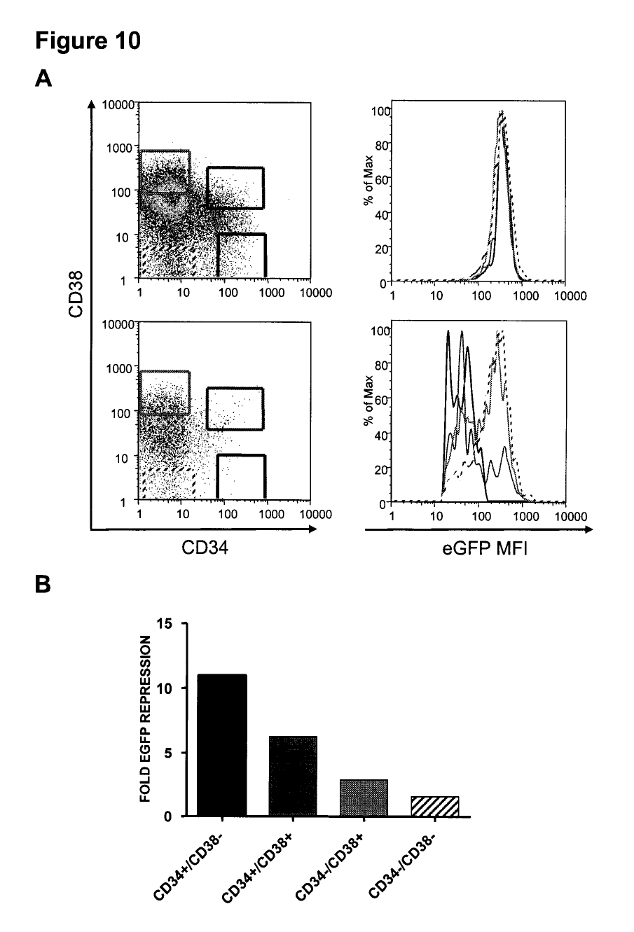

Referring to FIGS. 10A and 10B, the results reveal that levels of bioactive miR-126 were 11 fold higher in the CD34+38−(LSC-enriched) fraction of primary leukemia than in more mature subpopulations.

### Example 6

The in vivo biosensor-mediated expression of miR-126 in primary human CB after engraftment in a NOD/SCID mouse was investigated.

### Procurement and Lineage Depletion of Umbilical Cord Blood

In order to elucidate differential miRNA expression patterns within the CB hierarchy, miRNA expression profiling on FACS sorted CB subpopulations was performed. CB samples were obtained from placental and umbilical tissues according to procedures at the University Health Network (Toronto, ON). Samples were collected in heparin and centrifuged on Ficoll-Paque (Pharmacia, Uppsala) to obtain mononuclear cells. Lineage depletion and CD34+ enrichment were achieved by StemSep™ negative selection, according to the manufacturer's protocol (Stem Cell Technologies, Vancouver). The antibody cocktail specifically removes cells that express glycophorin A, CD2, CD3, CD14, CD16, CD19, CD24, CD41, CD56 or CD66b. The efficiency of primitive CD34+ cell enrichment was determined by flow cytometric analysis and Lin-CB cells were stored at −170° C. in 10% DMSO and 40% fetal bovine serum (McKenzie et al., 2006).

Bulk lin-CB cells were transduced with control and miR-126 Bd.LV.mirT lentivirus at an MOI of 50 for 72 hours in gene transfer conditions X-VIVO 10 media supplemented with 1% BSA and a cytokine cocktail including 10 μg/mL IL-6, 100 μg/mL SCF, 100 μg/mL FLT-3L, 10 μg/mL G-CSF and 15 μg/mL TPO. An equivalent of 3×10⁴ cells were injected into preconditioned NOD/SCID mice as previously described in FIG. 2. Ten weeks later, mice were euthanized and bone marrow harvested for analysis. Human lin-CB cells were enriched away from the murine bone marrow cells and human lineage positive cells by negative selection. Murine depletion and lin-CB cell enrichment were achieved by the combination of StemSep™ mouse/human chimera negative selection cocktail and StemSep™ Human hematopoietic progenitor cocktail (described above) according to the manufacturer's protocol (Stem Cell Technologies, Vancouver). Purified human lin-CB cells were then stained with a panel of antibodies against CD34, CD38, CD90, CD45RA, and NGFR as previously described in Example 2 and analyzed by multicolor flow cytometry.

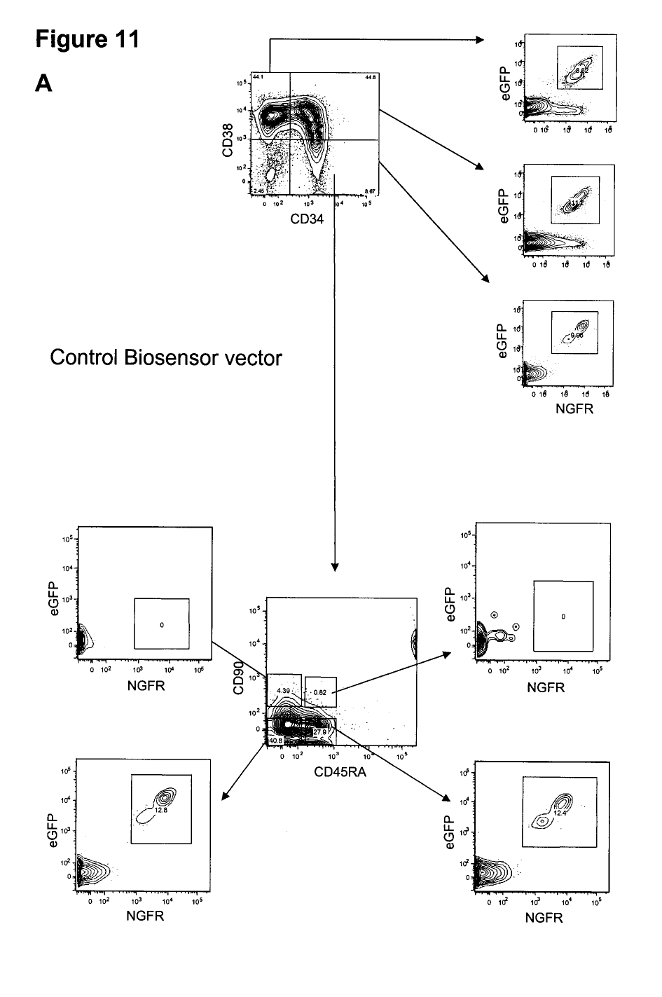

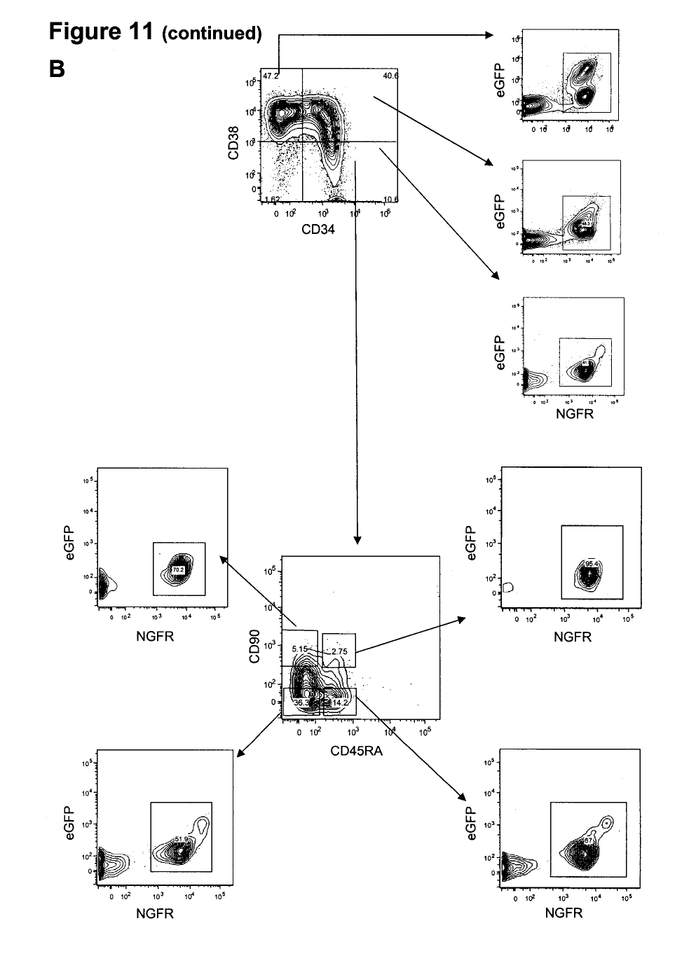

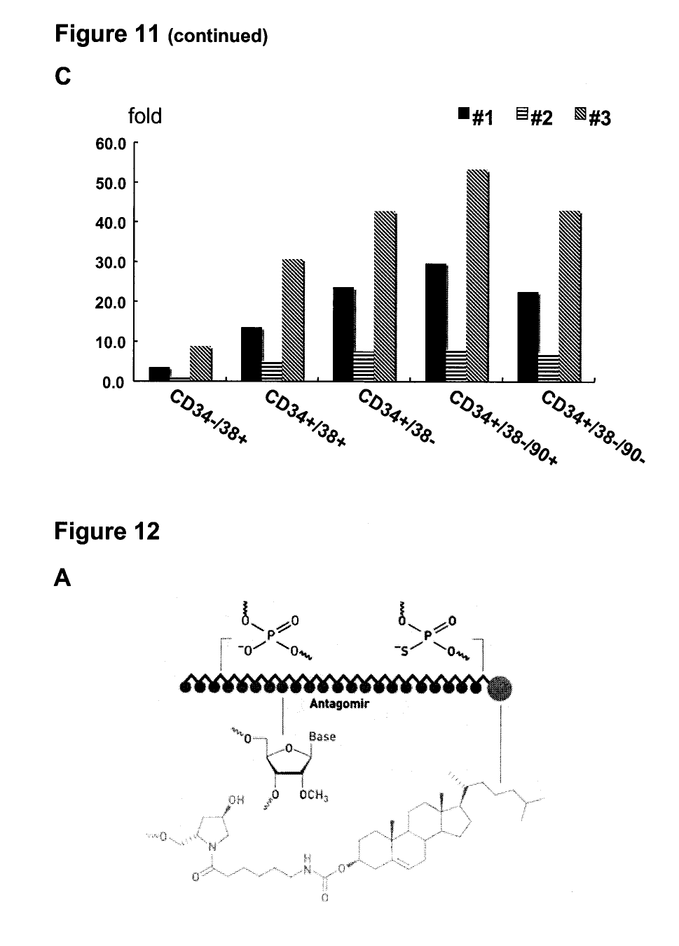

FIGS. 11A-C show that miR-126 activity was highest in the CD34+/CD38−/CD90+/CD45RA− fraction. These results show that the normal hematopoietic hierarchy displays very high miR-126 bioactivity (over 50 fold repression of eGFP) in the most highly enriched HSC compartment. In this compartment, as few as 10 cells are able to engraft a NOD/SCID immunodeficient mouse. Application of biosensor lentivectors for miR-126 in combination with existing cell surface HSC markers has the potential to further refine the purity of these cells.

### Example 7

Example 7 illustrates the specificity of the miR-126 biosensor lentivirus using antagomirs.

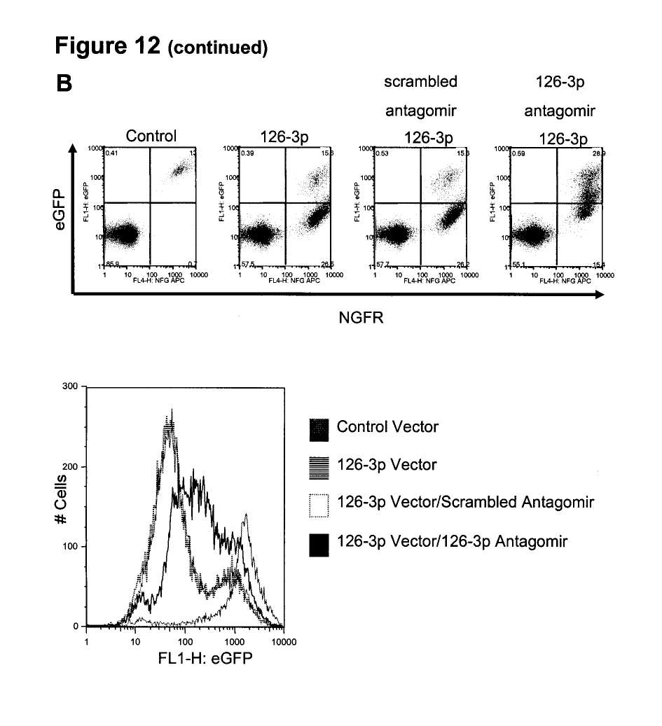

FIG. 12A shows the structure of antagomirs, small antisense RNA oligonucleotides designed to knockdown expression of specific miRNAs. Antagomir synthesis and use in culture is described below.

Single-stranded RNAs were custom synthesized as follows: 5′-cₛgₛcauuauuacucacgguaₛcₛgₛaₛ-chol-3′ for anti miR-126-3p and 5′-gₛuₛccuuaucauccaacguaₛcₛaₛaₛ-chol-3′ for the scrambled control antagomir. (Dharmacon, CO). The lower case letters represent 2′OMe-modified nucleotides: subscript ‘s’ represents a phosphorothioate linkage and chol represents cholesterol linked through a hydroxyprolinol linkage. Antagomirs were de-protected and added to a final concentration of 2 nM in serum free culture conditions once every seven days with cell passage as previously described for lin-CB and AML (Krutzfeldt et al., 2005).

Referring to FIG. 12B, FACS plots show antagomir-mediated knockdown of miR-126 within Bd.LV.miR-126-3pT transduced lin-CB. Antagomir mediated knockdown reverses eGFP repression by biologically active miR-126 with no effect from a scrambled antagomir control, evidencing that miR-126 repression of fluorescence from the miR-126 biosensor lentivirus is specific.

### Example 8

The prospective isolation of human CB derived HSC from long-term cultured lin-CB was investigated.

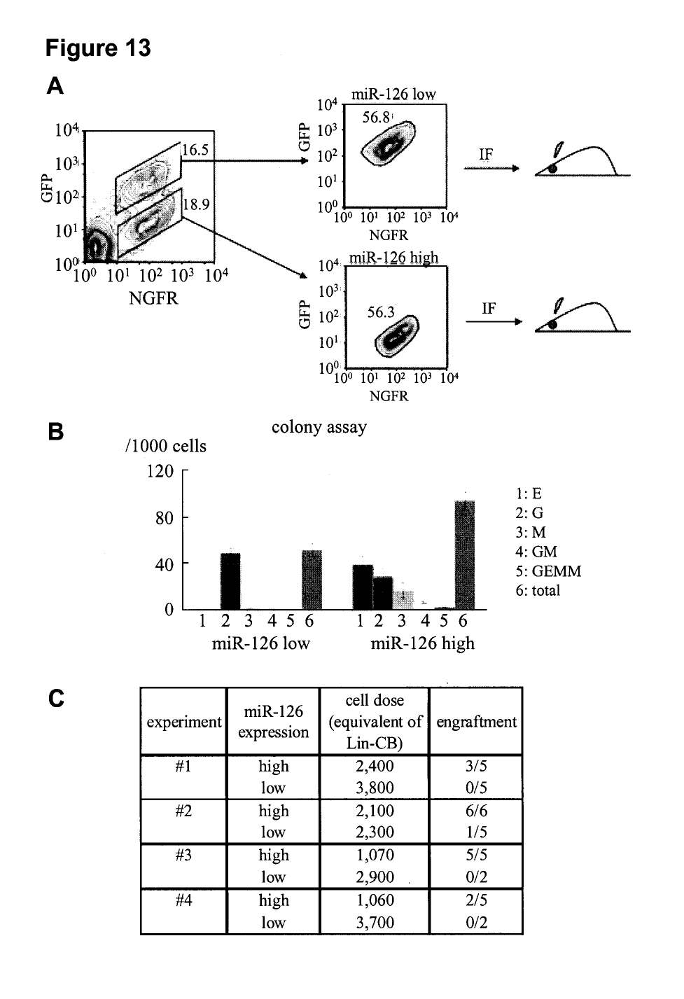

To evaluate the utility of miR-126 as a biomarker for primitive hematopoietic cells, Lin-CB cells were transduced with miRNA-126 Bd.LV and kept in culture in serum free liquid conditions for 2 weeks described in Example 2. Then, miRNA-126 high and miRNA-126 low populations were sorted as described in Example 1 and subject to colony assay and transplanted into immunodeficient mice as described in Example 2 (FIG. 13A). After 2 weeks of culture, surface antigens useful for purifying HSCs such as CD34, CD38, and CD90 are lost.

The data show that the miRNA-126^{low} fraction gave rise to only G/GM colonies (FIG. 13B; E: erythroid, G: granulocytic, M: macrophage, GM: granulocytic/macrophage, GEMM: granulocytic/erythroid/megakaryocyte/macrophage) and was unable to engraft NOD/SCID mice (FIG. 13C), whereas the miRNA-126^{high} fraction gave rise to all types of colonies (FIG. 13B) and successfully engrafted in mice with multi-lineage blood cells (FIG. 13C). Taken together, this data shows that high levels of miR-126 correlate with HSC/progenitor populations from cultured lin-CB.

### Example 9

The in vivo biosensor-mediated prospective isolation of LSC from primary AML after engraftment in a NOD/SCID mouse was investigated.

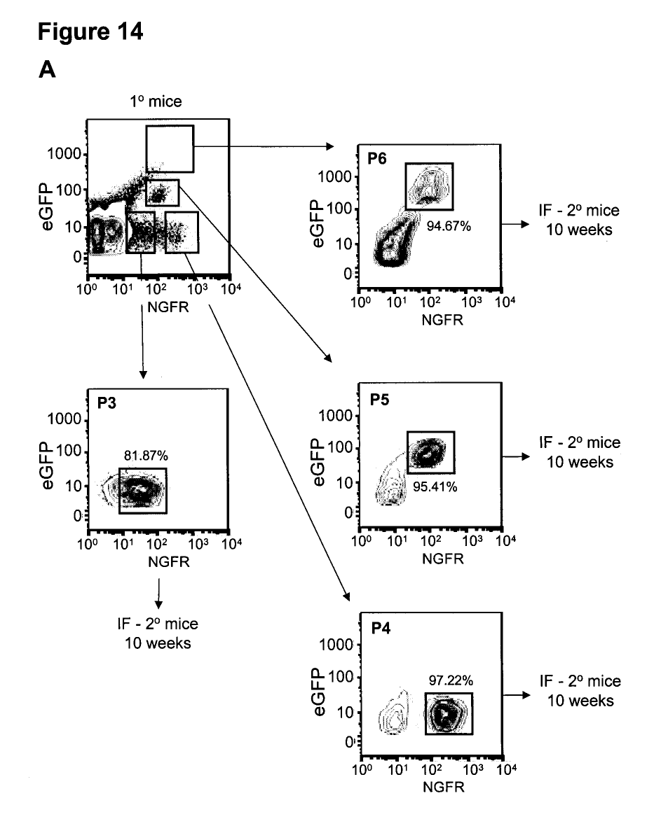

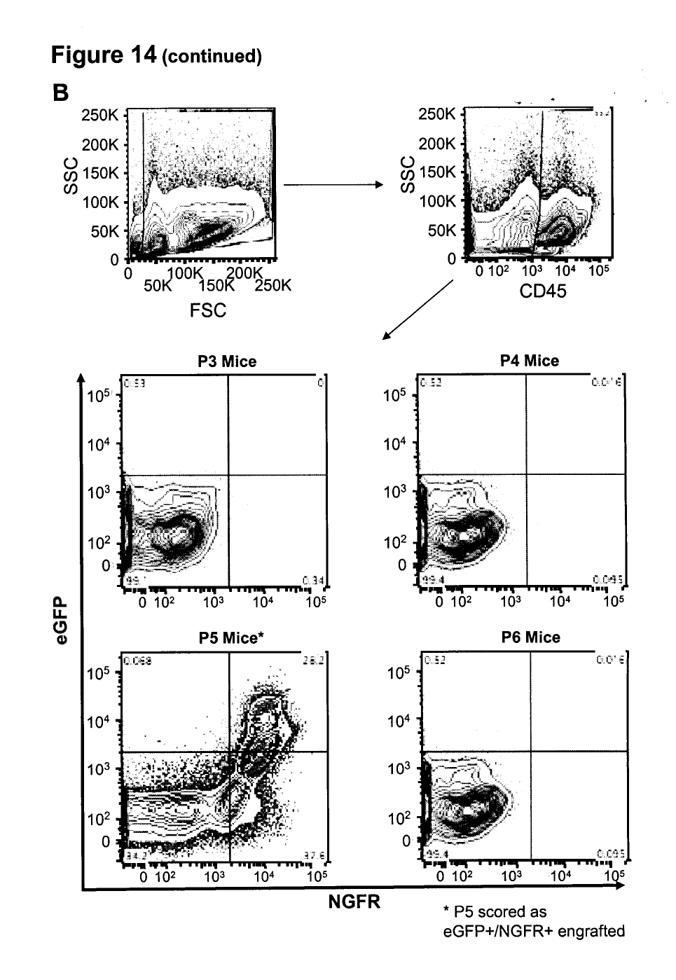

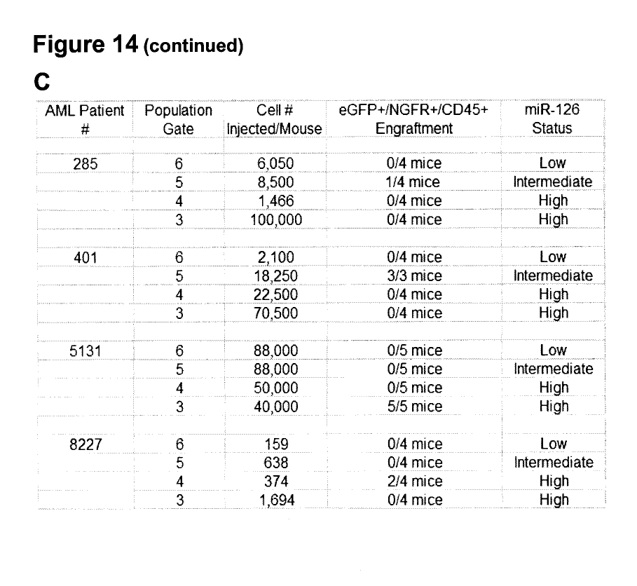

Sorted primary AML CD34+CD38− cells or bulk AML cells were transduced with control and miR-126 Bd.LV.mirT lentivirus at an MOI of 50 for 24-48 hours in standard AML culture conditions (FIG. 2). A pre-transduction equivalent of 4×10⁴−3×10⁵ cells were injected into preconditioned NOD/SCID mice as previously described in Example 2. Ten weeks later, mice were euthanized and bone marrow harvested for analysis. Human AML cells were enriched away from the murine bone marrow cells by negative selection. Murine depletion and AML cell enrichment were achieved by StemSep™ mouse/human chimera negative selection cocktail, according to the manufacturer's protocol (Stem Cell Technologies, Vancouver). Purified human AML cells were then stained with antibodies against CD45, CD34, CD38, and NGFR as previously described in Example 2. Fractions staining positive for NGFR and High/low/absent eGFP were sorted by high speed cell sorting and assessed for engraftment potential within 10 week secondary NOD/SCID repopulation assays (FIG. 14A). Mice were euthanized 10 weeks post-transplant and stained with antibodies against CD45, CD34, CD38, CD33, CD3, CD19 and NGFR to determine leukemia engraftment by flow cytometry. Secondary repopulation was scored positive only if the original eGFP/NGFR hierarchy was recapitulated in secondary mice (FIG. 14B.)

Referring to FIGS. 14A-C, the results show that high/intermediate levels of bioactive miR-126 mark LSC and all leukemia engraftment activity was contained within one gated population. These data are remarkable in that this was accomplished without the use of any classical LSC cell surface markers. Indeed, in most cases LSC for these patient samples were previously found within multiple sorting gates using the classical cell surface markers. This approach represents a leap forward in the ability to further purify the LSC.

### Example 10

Sensor vector based sorting of rare subpopulations of in vivo xenotransplanted lin-CB is anticipated. miRNA 126, identified in our screen, displayed high or unique expression within the normal HSC/progenitor fractions. Biosensor lentivectors engineered to contain specific miR-126 recognition motifs in the 3′ untranslated region of eGFP will be used to infect bulk lin-CB. Transduced cells will be cultured for 48-72 hours in minimal media conditions designed to preserve the primitiveness of the lin-CB and transplanted into immune-deficient NOD/SCID mice for 10 weeks. Cells recovered from the bone marrow of engrafted mice will be enriched by negative selection over a magnetic column and stained with antibodies to anti-human NGF receptor and other lin-CB stem cell marker combinations. Fractions that stain positive for NGFR and low/absent eGFP will be sorted by high speed cell sorting in combination with known normal and AML associated cell surface markers. To determine if enrichment of the HSC fraction has occurred, these sorted populations will be tested in limiting dilution within secondary NOD/SCID repopulation assays. We envision at least a two-fold enrichment of normal cord blood derived HSC using this sorting scheme. Further confirmation of the specificity of miR-126 in this context will be purification of HSC lacking classical stem cell markers.

Although preferred embodiments of the invention have been described herein, it will be understood by those skilled in the art that variations may be made thereto without departing from the spirit of the invention or the scope of the appended claims. All documents disclosed herein are incorporated by reference.

### REFERENCE LIST

Abbott, A. L., Alvarez-Saavedra, E., Miska, E. A., Lau, N. C., Bartel, D. P., Horvitz, H. R., and Ambros, V. (2005). The let-7 MicroRNA family members mir-48, mir-84, and mir-241 function together to regulate developmental timing in Caenorhabditis elegans. Dev. Cell 9, 403-414.
Ailles, L. E., Gerhard, B., and Hogge, D. E. (1997). Detection and characterization of primitive malignant and normal progenitors in patients with acute myelogenous leukemia using long-term coculture with supportive feeder layers and cytokines. Blood 90, 2555-2564.
Ambros, V., Lee, R. C., Lavanway, A., Williams, P. T., and Jewell, D. (2003). MicroRNAs and other tiny endogenous RNAs in C. elegans. Curr. Biol. 13, 807-818.
Andl, T., Murchison, E. P., Liu, F., Zhang, Y., Yunta-Gonzalez, M., Tobias, J. W., Andl, C. D., Seykora, J. T., Hannon, G. J., and Millar, S. E. (2006). The miRNA-processing enzyme dicer is essential for the morphogenesis and maintenance of hair follicles. Curr. Biol. 16, 1041-1049.
Bashirullah, A., Pasquinelli, A. E., Kiger, A. A., Perrimon, N., Ruvkun, G., and Thummel, C. S. (2003). Coordinate regulation of small temporal RNAs at the onset of Drosophila metamorphosis. Dev. Biol. 259, 1-8.
Bernstein, E., Caudy, A. A., Hammond, S. M., and Hannon, G. J. (2001). Role for a bidentate ribonuclease in the initiation step of RNA interference. Nature 409, 363-366.
Bernstein, E., Kim, S. Y., Carmell, M. A., Murchison, E. P., Alcorn, H., Li, M. Z., Mills, A. A., Elledge, S. J., Anderson, K. V., and Hannon, G. J. (2003). Dicer is essential for mouse development. Nat. Genet. 35, 215-217.
Brennecke, J., Hipfner, D. R., Stark, A., Russell, R. B., and Cohen, S. M. (2003). bantam encodes a developmentally regulated microRNA that controls cell proliferation and regulates the proapoptotic gene hid in Drosophila. Cell 113, 25-36.
Cai, X., Hagedorn, C. H., and Cullen, B. R. (2004). Human microRNAs are processed from capped, polyadenylated transcripts that can also function as mRNAs. RNA. 10, 1957-1966.
Cai, X., Lu, S., Zhang, Z., Gonzalez, C. M., Damania, B., and Cullen, B. R. (2005). Kaposi's sarcoma-associated herpesvirus expresses an array of viral microRNAs in latently infected cells. Proc. Natl. Acad. Sci. U.S.A 102, 5570-5575.
Calin, G. A., Dumitru, C. D., Shimizu, M., Bichi, R., Zupo, S., Noch, E., Aldler, H., Rattan, S., Keating, M., Rai, K., Rassenti, L., Kipps, T., Negrini, M., Bullrich, F., and Croce, C. M. (2002). Frequent deletions and down-regulation of micro-RNA genes miR15 and miR16 at 13q14 in chronic lymphocytic leukemia. Proc. Natl. Acad. Sci. U.S. A 99, 15524-15529.
Chen, C. Z., Li, L., Lodish, H. F., and Bartel, D. P. (2004). MicroRNAs modulate hematopoietic lineage differentiation. Science 303, 83-86.
Chen, X. (2005). microRNA biogenesis and function in plants. FEBS Lett.
Cimmino, A., Calin, G. A., Fabbri, M., Iorio, M. V., Ferracin, M., Shimizu, M., Wojcik, S. E., Aqeilan, R. I., Zupo, S., Dono, M., Rassenti, L., Alder, H., Volinia, S., Liu, C. G., Kipps, T. J., Negrini, M., and Croce, C. M. (2005). miR-15 and miR-16 induce apoptosis by targeting BCL2. Proc. Natl. Acad. Sci. U.S. A 102, 13944-13949.
Costinean, S., Zanesi, N., Pekarsky, Y., Tili, E., Volinia, S., Heerema, N., and Croce, C. M. (2006). Pre-B cell proliferation and lymphoblastic leukemia/high-grade lymphoma in E(mu)-miR155 transgenic mice. Proc. Natl. Acad. Sci. U.S. A 103, 7024-7029.
Eis, P. S., Tam, W., Sun, L., Chadburn, A., Li, Z., Gomez, M. F., Lund, E., and Dahlberg, J. E. (2005). Accumulation of miR-155 and BIC RNA in human B cell lymphomas. Proc. Natl. Acad. Sci. U.S. A 102, 3627-3632.
Harfe, B. D., McManus, M. T., Mansfield, J. H., Hornstein, E., and Tabin, C. J. (2005). The RNaseIII enzyme Dicer is required for morphogenesis but not patterning of the vertebrate limb. Proc. Natl. Acad. Sci. U.S. A 102, 10898-10903.
Harris, K. S., Zhang, Z., McManus, M. T., Harfe, B. D., and Sun, X. (2006). Dicer function is essential for lung epithelium morphogenesis. Proc. Natl. Acad. Sci. U.S. A 103, 2208-2213.
He, L., Thomson, J. M., Hemann, M. T., Hernando-Monge, E., Mu, D., Goodson, S., Powers, S., Cordon-Cardo, C., Lowe, S. W., Hannon, G. J., and Hammond, S. M. (2005). A microRNA polycistron as a potential human oncogene. Nature 435, 828-833.
Houbaviy, H. B., Murray, M. F., and Sharp, P. A. (2003). Embryonic stem cell-specific MicroRNAs. Dev. Cell 5, 351-358.
Jin, P., Alisch, R. S., and Warren, S. T. (2004). RNA and microRNAs in fragile X mental retardation. Nat. Cell Biol. 6, 1048-1053.
Johnson, S. M., Lin, S. Y., and Slack, F. J. (2003). The time of appearance of the C. elegans let-7 microRNA is transcriptionally controlled utilizing a temporal regulatory element in its promoter. Dev. Biol. 259, 364-379.
Khvorova, A., Reynolds, A., and Jayasena, S. D. (2003). Functional siRNAs and miRNAs exhibit strand bias. Cell 115, 209-216.
Krutzfeldt, J., Rajewsky, N., Braich, R., Rajeev, K. G., Tuschl, T., Manoharan, M., and Stoffel, M. (2005). Silencing of microRNAs in vivo with ‘antagomirs’. Nature 438, 685-689.
Lagos-Quintana, M., Rauhut, R., Lendeckel, W., and Tuschl, T. (2001a). Identification of novel genes coding for small expressed RNAs. Science 294, 853-858.
Lagos-Quintana, M., Rauhut, R., Lendeckel, W., and Tuschl, T. (2001b). Identification of novel genes coding for small expressed RNAs. Science 294, 853-858.
Lapidot, T., Sirard, C., Vormoor, J., Murdoch, B., Hoang, T., Caceres-Cortes, J., Minden, M., Patterson, B., Caligiuri, M. A., and Dick, J. E. (1994). A cell initiating human acute myeloid leukemia after transplantation into SCID mice. Nature 367, 645-648.
Lau, N. C., Lim, L. P., Weinstein, E. G., and Bartel, D. P. (2001a). An abundant class of tiny RNAs with probable regulatory roles in Caenorhabditis elegans. Science 294, 858-862.
Lau, N. C., Lim, L. P., Weinstein, E. G., and Bartel, D. P. (2001b). An abundant class of tiny RNAs with probable regulatory roles in Caenorhabditis elegans. Science 294, 858-862.
Lee, Y., Ahn, C., Han, J., Choi, H., Kim, J., Yim, J., Lee, J., Provost, P., Radmark, O., Kim, S., and Kim, V. N. (2003). The nuclear RNase III Drosha initiates microRNA processing. Nature 425, 415-419.
Lee, Y. S., Nakahara, K., Pham, J. W., Kim, K., He, Z., Sontheimer, E. J., and Carthew, R. W. (2004). Distinct roles for Drosophila Dicer-1 and Dicer-2 in the siRNA/miRNA silencing pathways. Cell 117, 69-81.
Liu, C. G., Calin, G. A., Volinia, S., and Croce, C. M. (2008). MicroRNA expression profiling using microarrays. Nat. Protoc. 3, 563-578.
Lund, E., Guttinger, S., Calado, A., Dahlberg, J. E., and Kutay, U. (2004). Nuclear export of microRNA precursors. Science 303, 95-98.
McKenzie, J. L., Gan, O. I., Doedens, M., Wang, J. C., and Dick, J. E. (2006). Individual stem cells with highly variable proliferation and self-renewal properties comprise the human hematopoietic stem cell compartment. Nat. Immunol. 7, 1225-1233.
Rodriguez, A., Griffiths-Jones, S., Ashurst, J. L., and Bradley, A. (2004). Identification of mammalian microRNA host genes and transcription units. Genome Res. 14, 1902-1910.
Tam, W., Ben Yehuda, D., and Hayward, W. S. (1997). bic, a novel gene activated by proviral insertions in avian leukosis virus-induced lymphomas, is likely to function through its noncoding RNA. Mol. Cell. Biol. 17, 1490-1502.
Tam, W., Hughes, S. H., Hayward, W. S., and Besmer, P. (2002). Avian bic, a gene isolated from a common retroviral site in avian leukosis virus-induced lymphomas that encodes a noncoding RNA, cooperates with c-myc in lymphomagenesis and erythroleukemogenesis. J. Virol. 76, 4275-4286.
Wang, Y., Medvid, R., Melton, C., Jaenisch, R., and Blelloch, R. (2007). DGCR8 is essential for microRNA biogenesis and silencing of embryonic stem cell self-renewal. Nat. Genet. 39, 380-385.
Xu, P., Vernooy, S. Y., Guo, M., and Hay, B. A. (2003). The Drosophila microRNA Mir-14 suppresses cell death and is required for normal fat metabolism. Curr. Biol. 13, 790-795.
Yi, R., Qin, Y., Macara, I. G., and Cullen, B. R. (2003). Exportin-5 mediates the nuclear export of pre-microRNAs and short hairpin RNAs. Genes Dev. 17, 3011-3016.

1. A method for identifying the engraftment potential of a population of hematopoietic stem cells (HSCs) comprising determining the relative level of miR-126 in the population, wherein increase in the relative level of miR-126 in the population is indicative of increased engraftment potential.

2. The method according to claim 1, further comprising sorting the population of HSCs into fractions
wherein the relative level of miR-126 in one or more fractions is determined; and
increase in the relative level of miR-126 in any of the one or more fractions is indicative of engraftment potential of that fraction.

3. The method of claim 2, wherein the population of HSCs is sorted using one or more biological markers.

4. The method of claim 3, wherein the one or more biological markers is selected from the group consisting of CD34, CD38, CD90 and CD45RA.

5. A method for increasing engraftment potential of a population of HSCs to be administered to a patient comprising:
a) sorting the population of HSCs into fractions; and
b) selecting fractions exhibiting increased levels of miR-126 expression for administration to the patient.

6. The method of claim 5, wherein the population of HSCs is sorted using one or more biological markers.

7. The method of claim 6, wherein the one or more biological markers is selected from the group consisting of CD34, CD38, CD90 and CD45RA.

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A method for purifying HSCs from a population of cells comprising:
a) sorting the population of cells into fractions; and
b) selecting fractions exhibiting increased levels of miR-126 expression.

22. The method of claim 21, wherein the population of HSCs is sorted using one or more biological markers.

23. The method of claim 22, wherein the one or more biological markers is selected from the group consisting of CD34, CD38, CD90 and CD45RA.

24. The method of claim 23, wherein the fraction exhibiting increased levels of miR-126 expression is a CD34+ fraction.

25. The method of claim 24, wherein the fraction exhibiting increased levels of miR-126 expression is a CD38− fraction.

26. The method of claim 25, wherein the fraction exhibiting increased levels of miR-126 expression is a CD90+ fraction.

27. The method of claim 26, wherein the fraction exhibiting increased levels of miR-126 expression is a CD45RA− fraction.


SEQUENCE LISTING (1 sequence)

SEQ ID NO: 1 - RNA, 22 nt, Homo sapiens
ucguaccgugaguaauaaugcg